(19) United States
(12) Patent Application Publication
(10) Pub. No.: US 2005/0148963 A1
(43) Pub. Date: Jul. 7, 2005

(54) Bioabsorbable surgical sponge

(76) Inventor: H. George Brennan, Newport Beach, CA (US)

Correspondence Address: Michael G. Petit, P.O. Box 91929, Santa Barbara, CA, 93190-1929, US

(21) Appl. No.: 10/752,202

(22) Filed: Jan. 5, 2004

(51) Int. Cl. A61F 13/15; A61F 13/20

(52) U.S. Cl. 604/364

(57) ABSTRACT

A sterile, bioabsorbable, disposable surgical sponge operable for absorbing blood from within a surgical site. The sponge is an open cell porous body made from a bioabsorbable polymer, a carbohydrate, a protein or a hydrogel. In the event that the sponge is accidentally left within the body during a surgical procedure, the sponge is biodegraded and excreted by the body.

### BACKGROUND OF THE INVENTION

1. Field of the Invention

The present invention relates to surgical sponges used for the removal of blood from an operative site and, more particularly, to a biodegradable surgical sponge.

2. Prior Art

The unintentional implantation of a surgical sponge in an operative site within a patient is a common problem. Although it is the current practice for a surgical nurse to reconcile the sponge count before wound closure, inadvertent errors occur and a sponge may be left within a surgical site when the wound is closed. When the error is discovered, the surgeon must reexpose the surgical site and retrieve the sponge, causing undue further stress and trauma to the patient.

Surprisingly, although biodegradable materials have enjoyed use in wound dressings (i.e., an application wherein there is no danger of accidental implantation of a foreign body within a patient), the bioabsorbable materials and technology has not been extended for use in pledgets that are used for the intraoperative absorbtion of blood within the surgical site. There is, therefore, a current need for a surgical sponge that is biodegradable by enzymatic action within the body and wherein the biodegration products are either absorbed or excreted by the body without harm to the patient.

### SUMMARY

It is an object of the present invention to provide a disposable, bioabsorbable surgical sponge operable for absorbing blood from within a surgical wound.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is an elevational view of a preferred embodiment of a bioabsorbable surgical sponge in accordance with the present invention.

FIG. 2 is a cross-sectional view of a surgical sponge in accordance with the preferred embodiment of the present invention illustrated in FIG. 1.

FIG. 3 is an enlarged view of a portion of the surgical sponge of FIG. 2 showing the pores formed between the network of bioabsorbable polymer filaments.

### DESCRIPTION OF THE PREFERRED EMBODIMENTS

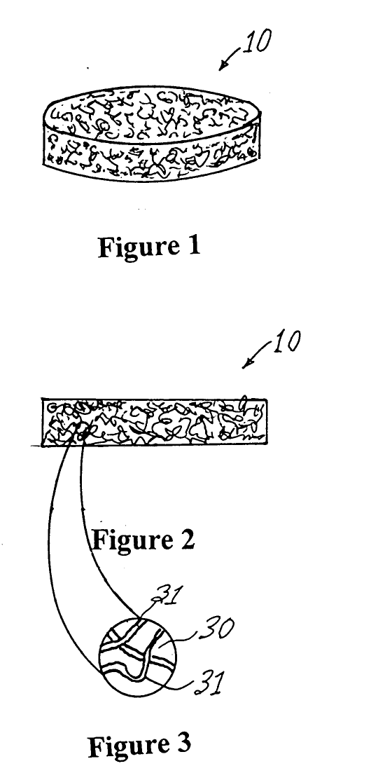

With reference now to FIGS. 1-3, a bioabsorbable surgical sponge is shown in perspective view at numeral 10 in FIG. 1 and in cross-sectional view in FIG. 2. An enlarged portion of a filamentous embodiment of the surgical sponge 10 is shown in FIG. 3. The sponge 10, though shown in the shape of a circular pad, may be formed in any desired and sensible shape. The sponge 10 is comprised of a bioabsorbable material formed into a body having a plurality of interconnecting pores 30 (FIG. 3) that open to the surface of the sponge. The bioabsorbable material may be a fibrous mass of bioabsorbable filaments 31 or it may be a bioabsorbable “open cell” foam body.

The biodegradable material used for the construction of the sponge should preferably have a hydrophilic outer surface to facilitate the absorbtion of blood into the sponge. Suitable biodegradable materials for fabricating the surgical sponge 10 include filaments or foam bodies comprised of polymers or copolymers of lactide, glycolide, caprolactone, polydioxanone, trimethylene carbonate, polyorthoesters and polyethylene oxide, collagen and high molecular weight polysaccharides from connective tissue such as chondroitin salts. Other polysaccharides that can be formed into a porous body may also prove suitable, such as chitin and chitosan. Additional bioabsorbable materials are in intense development and it is expected that many of the new materials will also be applicable for forming a biodegradable surgical sponge in accordance with the present invention.

A filamentous surgical sponge in accordance with the present invention may be fabricated by weaving the bioabsorbable polymer filaments into a sheet and cutting appropriately dimensioned pledgets therefrom. Alternatively, a bolus of filament can be placed into a cylinder heated to a temperature less than, but close to, the transition temperature of the polymer and compressed with a heated piston to form a circular pad as shown in FIGS. 1 and 2. The pressure applied to the piston can be varied to affect the appropriate pad thickness and desired average pore size. The sponge may be sterilized by heat, ethylene oxide or radiation, the choice depending on the bioabsorbable material selected.

The advantage of a surgical spong in accordance with the present invention is that in the event that the sponge is accidentally implanted within the body when a surgical wound is closed, the sponge will be biodegraded and the biodegradation products excreted from the body without the need for surgical explantation. The bioabsorbable surgical sponge reduces surgical complications due to foreign body response and obviates the need for further surgery thereby reducing trauma to a patient.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A bioabsorbable surgical sponge operable for the intraoperative absorbtion of blood from within an open surgical site within a patient, the surgical sponge comprising a porous body consisting of a bioabsorbable material.

2. The bioabsorbable surgical sponge of claim 1 wherein said bioabsorbable material is hydrophilic.

3. The bioabsorbable surgical sponge of claim 1 wherein said bioabsorbable material is selected from the group consisting of polymers or copolymers of lactide, glycolide, caprolactone, polydioxanone, trimethylene carbonate, polyorthoesters and polyethylene oxide, collagen, chondroitin, chitin and chitosan.
